(19) United States
(12) Patent Application Publication
(10) Pub. No.: US 2011/0256063 A1
(43) Pub. Date: Oct. 20, 2011

(54) MEX3C REGULATION AND TARGET TO CONTROL OBESITY AND DIABETES

(76) Inventors: Baisong Lu, Winston-Salem, NC (US); Colin E. Bishop, Clemmons, NC (US)

(21) Appl. No.: 12/952,526

(22) Filed: Nov. 23, 2010

Related U.S. Application Data
(60) Provisional application No. 61/263,535, filed on Nov. 23, 2009.

(51) Int. Cl. A61K 49/00; A61K 38/16; A61K 39/395; G01N 30/02; A61P 3/04; C12Q 1/02; G01N 33/566; G01J 3/42; A61K 31/7088; A61K 31/713

(52) U.S. Cl. 424/9.2; 514/44 R; 514/4.8; 424/130.1; 514/44 A; 435/29; 436/501; 356/319; 73/61.52

(57) ABSTRACT

MEX3C deficiency impairs the development of white and brown adipose tissue. Hence the present invention provides, among other things, a method of screening a candidate compound for activity in inhibiting fat deposition in a subject in need thereof and/or treating a condition in a subject in need thereof, comprising: (a) contacting a candidate compound to a cell that expresses MEX3C protein; and then (b) detecting a quantity of expression of the MEX3C protein in the cell; a depression in the expression of MEX3C protein when the candidate compound is contacted thereto as compared to that expressed when the candidate compound is not contacted thereto indicating the compound is active in inhibiting fat deposition and/or treating a condition in a subject in need thereof. Methods of treatment and screening subjects are also described.

### RELATED APPLICATIONS

This application claims the benefit of U.S. Provisional Patent Application Ser. No. 61/263,535, filed Nov. 23, 2009, the disclosure of which is incorporated by reference herein in its entirety.

### Government Support

This invention was made with Government support under grant numbers U01HD043421 and R01HD058058 from the National Institutes of Health. The United States Government has certain rights to this invention.

### BACKGROUND OF THE INVENTION

Obesity is epidemic worldwide with more than 1 billion adults overweight and at least 300 million clinically obese. It is a major contributor to the global burden of chronic disease and disability. Adipocytes are important in the control of energy balance and lipid homeostasis. In mammals there are two distinct types of adipocytes: white adipose tissue (WAT) and brown adipose tissue (BAT). The development of adipose tissue is controlled by a group of transcription factors among which peroxisome proliferator-activated receptor (PPAR) gamma and CCAAT/enhancer binding protein (C/EBP) family are the most important (Gesta et al., 2007). PPARγ plays an important role in adipogenesis and has been shown to be necessary and sufficient for adipocyte differentiation (Tontonoz et al., 1994).

Although WAT development occurs during late prenatal and early postnatal life, the existence of adipocyte precursors or progenitor cells in vivo enables WAT the ability to expand during adult life when energy intake exceeds energy expenditure. It is suggested that approximately 10% of the body's fat cells are regenerated each year (Spalding et al., 2008). Adipose progenitor cells make it possible to maintain adipose dynamics in adult. Adipose progenitor cells can be isolated from the adipose stroma-vascular fraction and these cells show high expression of the preadipocyte markers Pref1, GATA3, Wisp2, Smo, and Gli3 but lower levels of mature adipocyte markers (Tang et al., 2008). These precursor cells keep the proliferative capability and are capable of differentiating into mature adipocytes in vitro.

Numerous transgenic and knockout animal models have been generated which greatly enhanced our understanding of adipogenesis (Reue and Phan, 2006). For example, forced expression of a dominant negative protein to inhibit the function of B-ZIP proteins in both the C/EBP and AP-1 families of transcription factors ablates adipose tissue development at all (Moitra et al., 1998). Forced expression of Pparδ in adipose tissue prevents the development of obesity (Wang et al., 2003). More work is needed to fully understand the regulation of adipose development and energy homeostasis.

C. elegans MEX-3 is a KH domain-containing RNA binding protein. It is involved in the cell fate specification during early C. elegans embryonic stage and the maintenance of the totipotency of the germline in adult worms (Ciosk et al., 2006; Draper et al., 1996; Hunter and Kenyon, 1996; Musco et al., 1996). Human and mouse genomes encode four MEX-3 homologues, named MEX3A, MEX3B, MEX3C and MEX3D (Buchet-Poyau et al., 2007). The in vivo functions of mammalian mex-3 homologues are not yet explored.

MEX3C (once called RKHD2) is highly conserved among mammalian species. Human, chimpanzees, mouse and cow MEX3Cs are 99% identical when the C-terminal 460 amino acids are compared. A combination of sibling-pair linkage analysis and case-control association studies suggests a contribution of MEX3C to the genetic susceptibility of hypertension although any possible mechanism is unknown (Guzman et al., 2006). The possible in vivo function of Mex3c has not been clear.

### SUMMARY OF THE INVENTION

Here we report that MEX3C deficiency impairs the development of white and brown adipose tissue. MEX3C deficiency leads to less adipose tissue deposition without affecting the other internal organs. Mex3c mutation impairs the adipocyte differentiation capacity of adipose progenitor cells isolated from adipose tissue. Although MEX3C deficiency reduces the adipose tissue mass, it does not affect food intake, lipid absorption or blood glucose, which suggests that energy expenditure in the mutants is increased. Among other things, our data indicate that MEX3C is a new drug target for obesity control and prevention/treatment of diabetes.

A first aspect of the invention is a method of screening a candidate compound for activity in inhibiting fat deposition in a subject in need thereof and/or treating a condition in a subject in need thereof, comprising: (a) contacting a candidate compound to a cell that expresses MEX3C protein; and then (b) detecting a quantity of expression of the MEX3C protein in the cell; a depression in the expression of MEX3C protein when the candidate compound is contacted thereto as compared to that expressed when the candidate compound is not contacted thereto indicating the compound is active in inhibiting fat deposition and/or treating a condition in a subject in need thereof.

A further aspect of the invention is a method of screening a candidate compound for activity in inhibiting fat deposition in a subject in need thereof and/or treating a condition in a subject in need thereof, comprising: (a) contacting a candidate compound to a MEX3C protein in vitro; and then (b) detecting binding (e.g., by competitive binding assay) of the MEX3C protein to the candidate compound, binding of the candidate compound to the MEX3C protein indicating the candidate compound is active in inhibiting fat deposition and/or treating a condition in a subject in need thereof.

A further aspect of the invention is a method of screening a subject for a predisposition to a metabolic disorder (e.g., obesity, diabetes, etc.), comprising: detecting elevated expression of MEX3C protein in a biological sample from the subject; and determining the subject is predisposed to a metabolic disorder from the detected elevated expression.

In some embodiments of the foregoing, the cell is in vitro. In some embodiments of the foregoing, the contacting step is carried out by adding the compound to a media carrying the cell.

In some embodiments of the foregoing, the cell is in vivo in a host animal.

In some embodiments of the foregoing, the contacting step is carried out by orally or parenterally administering the compound to the animal.

In some embodiments of the foregoing, the host cell is a mammalian host cell (which may reside in a mammalian subject when the method is carried out in vivo as noted above).

In some embodiments of the foregoing, the host cell or subject is a human, primate, cat, dog, rat, or mouse host cell.

In some embodiments of the foregoing, the cell is a nerve cell, smooth muscle cell, granulosa cell, early spermatogenic cells, or late haploid spermatids. The invention is not limited to these embodiments, as MEX3C is expressed in all cells.

In some embodiments of the foregoing, the detecting step further comprises at least partially purifying a sample of MEX3C protein from the cell.

In some embodiments of the foregoing, the detecting step further comprises immunologically, spectrophotometrically, and/or chromatographically detecting the MEX3C protein.

In some embodiments of the foregoing, the compound is selected from the group consisting of oligomers and non-oligomers.

In some embodiments of the foregoing, the host cell or MEX3C protein is mammalian (e.g., human, primate, cat, dog, rat, mouse, etc.)

In some embodiments of the foregoing, the biological sample is a blood, cell, or tissue sample.

In some embodiments of the foregoing, the method further comprises at least partially purifying the biological sample.

In some embodiments of the foregoing, the detecting step further comprises immunologically, spectrophotometrically, or chromatographically detecting the MEX3C protein.

Utility. The present invention is useful in identifying compounds that are candidates for treating, or for screening for further activity in treating, or as candidates for further screening for activity in treating, a variety variety of diseases, disorders and conditions, including but not limited to obesity, diabetes including type II diabetes, syndrome X, glaucoma, hyperlipidemia, hyperglycemia and hyperinsulinemia, vascular conditions associated with elevated sterol and/or stanol levels, arteriosclerotic disease, renal disease, hepatic disease, metabolic syndrome, etc.

In some embodiments, the present invention relates to the inhibition of MEX3c. In some embodiments, inhibition is inhibition of nucleic acid transcripts encoding MEX3c, for example inhibition of messenger RNA (mRNA). In alternative embodiments, inhibition of MEX3c is inhibition of the expression and/or inhibition of activity of the gene product of MEX3c, for example the polypeptide or protein of MEX3c, or isoforms thereof. As used herein, the term “gene product” refers to RNA transcribed from a gene, or a polypeptide encoded by a gene or translated from RNA.

In some embodiments, inhibition of MEX3c is by an agent. One can use any agent, for example but are not limited to nucleic acids, nucleic acid analogues, peptides, phage, phagemids, polypeptides, peptidomimetics, ribosomes, aptamers, antibodies, small or large organic or inorganic molecules, or any combination thereof. In some embodiments, agents useful in methods of the present invention include agents that function as inhibitors of MEX3c expression, for example inhibitors of mRNA encoding MEX3c.

### BRIEF DESCRIPTION OF THE DRAWINGS

FIG. 1. Targeting of Mex3c by a gene trap strategy.

FIG. 1A. Genomic structure and splicing of the Mex3c^{Gt(DD0642)Wtsi} allele. The black line indicates the genomic DNA, and the blue and green solid boxes indicate exons 1 and 2 of Mex3c. The two splice acceptor (SA) sites from the gene trap vector are in red (the SA site designed for the vector) and light blue (the cryptic SA site found in this study) respectively. Three different transcripts (splicing 1-3) can be generated from the trapped allele.

FIG. 1B. Detection of variant splicing in normal mice (+/+), heterozygous mice (+/tr) and homozygous gene trap mice (tr/tr). Only the normal Mex3c mRNA (splice variant 3) could be detected in +/+ mice. Three different transcripts can be detected in the heterozygous and homozygous gene trap mice. The normal Mex3c mRNA found in the homozygous gene trap mice is about 5-10% of that of +/+ mice.

FIG. 1C. Sequence of the splice junction of splice variant 1. Exon 1 of Mex3c (blue) was spliced into Lac-Z sequence (red) from the vector.

FIG. 1D. Sequence of the splice junction of splice variant 2. An encrypted exon from the gene trap vector (red) was inserted between exon 1 and exon 2 (blue) of Mex3c.

FIG. 1E. Expression of MEX3C protein in homozygous gene trap mice. Low level of MEX3C protein (˜10% of normal value) could be detected in the testis.

FIG. 2. Reduced adiposity in MEX3C-deficient mice. A. Mutant mice weighed less than normals, which was more pronounced at older age. Presented were data from female mice (n≧5 for each time point). B. Kidney and perirenal fat. Note the comparable kidney size and the smaller perirenal fat size. C. Other internal organs were unaffected in the mutants except white and brown adipose tissues. D. H&E staining of fat tissue sections showing hypotrophy and hypoplasia of mutant adipose tissues. In A and C, controls included mice of +/+ and +/tr since there was no significant difference in these parameters between +/+ and +/tr groups. Mean±SEM are presented. * indicates significant difference.

FIG. 3. Glucose maintenance and insulin sensitivity in MEX3C-deficient mice. A. MEX3C-deficient mice had reduced random blood glucose concentration (measured without fasting). B. MEX3C-deficient mice showed reduced fasting blood glucose concentrations. C. MEX3C-deficient mice showed improved blood glucose control in glucose tolerance test. D. MEX3C-deficient mice showed improved insulin sensitivity in insulin tolerance test. N=6 for all genotypes. Mice were 10-20 weeks old. Data shown as mean±SEM; * indicates p_{<}0.05 by ANOVA.

FIG. 4. Less triglyceride and glycogen content in tr/tr mouse livers. Top: Liver cryosections were stained by Oil Red 0. Oil drops were stained red, which is readily observed in control (+/+ and +/tr) liver but hardly visible in mutant liver. Compared with +/+ liver, +/tr liver also show less oil drops. Bottom: Reduced glycogen deposition in tr/tr mouse liver. Glycogen was stained red by periodic acid Schiff staining. Animals were 6 months when being sacrificed. Scale bar: 50 μm.

FIG. 5. Expression of Mex#c in various tissues as determined by RT-PCR.

FIG. 6. Body weight of control and MEX3C deficient mice on chow and high-fat diet. Mice started on high-fat diet from age of 5 weeks. Body weight was monitored weekly. Normal control mice started to show significantly larger body weight 5 weeks after on high-fat diet (n≧5 at each time point, ANOVA). MEX3C deficient mice on high-fat diet showed marginally increased body weight than MEX3C deficient mice on chow diet but the difference did not reach statistic significance. * indicates significant difference between normal control mice on chow and high-fat diet. No significance was observed between the body weight of MEX3C deficient mice on chow and high-fat diet. Data shown as mean±SEM.

FIG. 7. Organ of normal control and MEX3C deficient mice on different diets. Shown were organs from female mice of 17 weeks. Mice on high-fat diet were fed with this diet starting from the age of 5 weeks.

FIG. 8. Glucose maintenance of MEX3C deficient mice on high-fat diet. A. MEX3C deficient mice on high-fat diet did not develop glucose intolerance. B. MEX3C deficient mice on high-fat diet were highly sensitive to insulin. High-fat diet groups were assayed 12 weeks after they have been on this diet. Represented were means±SEM (n≧5).

### DETAILED DESCRIPTION OF EMBODIMENTS OF THE INVENTION

“Non-oligomers” as used herein include a wide variety of organic molecules, such as heterocyclics, aromatics, alicyclics, aliphatics and combinations thereof, comprising steroids, antibiotics, enzyme inhibitors, ligands, hormones, drugs, alkaloids, opioids, terpenes, porphyrins, toxins, catalysts, as well as combinations thereof.

“Oligomers” as used herein include oligopeptides, oligonucleotides, oligosaccharides, polylipids, polyesters, polyamides, polyurethanes, polyureas, polyethers, and poly (phosphorus derivatives), e.g. phosphates, phosphonates, phosphoramides, phosphonamides, phosphites, phosphinamides, etc., poly (sulfur derivatives) e.g., sulfones, sulfonates, sulfites, sulfonamides, sulfenamides, etc., where for the phosphorous and sulfur derivatives the indicated heteroatom for the most part will be bonded to C,H,N,O or S, and combinations thereof.

“Express” or “expression” of MEX3C means that a gene encoding MEX3C is transcribed, and preferably, translated. Typically, according to the present invention, expression of a MEX3C coding region will result in production of the encoded polypeptide, such that the cell is an “MEX3C producing cell.” In some embodiments, cells produce MEX3C without further manipulation such as the introduction of an exogenous gene. In other embodiments, cells are induced to expression MEX3C, e.g., by transient or constitutive expression directed by an introduced exogenouse gene comprising a MEX3C coding region, e.g., in cell lines such as 293 cells, COS cells, CHO cells, fibroblasts, and the like, genetically engineered to express the MEX3C for screening purposes.

“Active agent” as used herein refers to any entity which is normally not present or not present at the levels being administered in the cell. The agent can in some embodiments be selected from the group consisting of chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein inhibitor of MEX3c within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds. See, e.g., US Patent Application Publication No. 2010 0239565

“Nucleic acid” or “oligonucleotide” or “polynucleotide” as used herein can mean at least two nucleotides covalently linked together. As will be appreciated by those in the art, the depiction of a single strand also defines the sequence of the complementary strand. Thus, a nucleic acid also encompasses the complementary strand of a depicted single strand. As will also be appreciated by those in the art, many variants of a nucleic acid can be used for the same purpose as a given nucleic acid. Thus, a nucleic acid also encompasses substantially identical nucleic acids and complements thereof. As will also be appreciated by those in the art, a single strand provides a probe for a probe that can hybridize to the target sequence under stringent hybridization conditions. Thus, a nucleic acid also encompasses a probe that hybridizes under stringent hybridization conditions. Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. A nucleic acid will generally contain phosphodiester bonds, although nucleic acid analogs can be included that can have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides, containing a non naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7 deaza-adenosine; O- and N-alkylated nucleotides, e.g. N6-methyl adenosine are suitable. The 2′ OH— group can be replaced by a group selected from H. OR, R. halo, SH, SR, NH2, NHR, NR2 or CN, wherein R is C—C6 alkyl, alkenyl or alkynyl and halo is F, Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

“Inhibiting” as used herein as it pertains to the expression or activity of MEX3c does not necessarily mean complete inhibition of expression and/or activity. Rather, expression or activity of the protein, polypeptide or polynucleotide is inhibited to an extent, and/or for a time, sufficient to produce the desired effect. In particular, inhibition of MEX3c can be determined using an assay for MEX3c inhibition, for example but not limited to using the bioassay for MEX3c protein as disclosed herein. Agents that inhibit MEX3c are agents that inhibit the MEX3c protein and/or MEX3c function by at least 10%. In some embodiments, an inhibitor of MEX3c is an agent that inhibits MEX3c protein or expression of MEX3c by at least 10%.

“Treating” as used herein includes reducing or alleviating at least one adverse effect or symptom of a condition, disease or disorder associated with undesired weight gain.

“Effective amount” as used herein refers to the amount of therapeutic agent of pharmaceutical composition to provide “effective” treatment as that term is defined herein. An effective amount as used herein would also include an amount sufficient to prevent or delay the development of a symptom of the disease, alter the course of a symptom disease (for example but not limited to, slow the progression of a symptom of the disease), or reverse a symptom of the disease.

“Administering,” and “introducing” as used herein are used interchangeably and refer to the placement of the agents that inhibit MEX3c as disclosed herein into a subject by a method or route which results in at least partial localization of the agents at a desired site. The compounds of the present invention can be administered by any appropriate route which results in an effective treatment in the subject. Examples include but are not limited to oral, transcutaneous, inhalation, and parenteral (e.g., intraveneous, intramuscular, intraperitoneal) administration.

“Subject,” “individual” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

“Gene” as used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

“Gene silencing” or “gene silenced” as used herein in reference to an activity of n RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

“RNAi” as used herein refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

“siRNA” as used herein refers to a nucleic acid that forms a double stranded RNA, which double stranded RNA has the ability to reduce or inhibit expression of a gene or target gene when the siRNA is present or expressed in the same cell as the target gene, for example MEX3c. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length). As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

“microRNA” or “miRNA” as used herein are used interchangeably herein are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways. As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

The present invention may be carried out in accordance with known techniques or variations thereof that will be apparent to those skilled in the art. See, e.g., U.S. Pat. No. 5,908,609.

Any suitable means for screening MEX3C expression may be used to carry out the present invention, including but limited to chromatography, immunoassay, and polymerase chain reaction. Kits may be provided that provide appropriate reagents (e.g., antibodies that specifically bind to the MEX3C protein; PCR primers for detection of MEX3C expression, buffer solutions, etc., optionally including instructions for carrying out the screening method and optionally packaged together in a common container.

A variety of agents from various sources can be screened for activity by using the methods of the present invention. Agents to be screened can be naturally occurring or synthetic molecules. Agents to be screened can also obtained from natural sources, such as, e.g., marine microorganisms, algae, plants, fungi, etc. Alternatively, agent to be screened can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Agents can include, e.g., pharmaceuticals, therapeutics, environmental, agricultural, or industrial agents, pollutants, cosmeceuticals, drugs, organic compounds, lipids, glucocorticoids, antibiotics, peptides, proteins, sugars, carbohydrates, chimeric molecules, etc. (see, e.g., U.S. Pat. No. 7,041,276).

Compound libraries or combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the National Cancer Institute's (NCI) Natural Product Repository, Bethesda, Md., the NCI Open Synthetic Compound Collection, Bethesda, Md., NCI's Developmental Therapeutics Program, or the like.

The compounds which may be screened in accordance with the invention include, but are not limited to peptides, antibodies and fragments thereof, and other organic compounds (e.g., peptidomimetics) that bind to the MEX3C or a portion thereof.

Compounds may include, but are not limited to, peptides such as, for example, soluble peptides, including but not limited to members of random peptide libraries; (see, e.g., Lam, K. S. et al., 1991, Nature 354: 82-84; Houghten, R. et al., 1991, Nature 354: 84-86), and combinatorial chemistry-derived molecular library made of D- and/or L-configuration amino acids, phosphopeptides (including, but not limited to, members of random or partially degenerate, directed phosphopeptide libraries; see, e.g., Songyang, Z. et al., 1993, Cell 72: 767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Other compounds which can be screened in accordance with the invention include but are not limited to small organic molecules that are able to cross the blood-brain barrier, gain entry into an appropriate cell and affect the expression of the MEX3C gene or some other gene involved in the MEX3C signal transduction pathway (e.g., by interacting with the regulatory region or transcription factors involved in gene expression); or such compounds that affect the activity of the MEX3C or the activity of some other intracellular factor involved in the MEX3C signal transduction pathway.

Computer modelling and searching technologies permit identification of compounds, or the improvement of already identified compounds, that can modulate MEX3C expression or activity. Having identified such a compound or composition, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modelling can be used to complete the structure or improve its accuracy. Any recognized modelling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential MEX3C modulating compounds.

Alternatively, these methods can be used to identify improved modulating compounds from an already known modulating compound or ligand. The composition of the known compound can be modified and the structural effects of modification can be determined using the experimental and computer modelling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of MEX3C and related transduction and transcription factors will be apparent to those of skill in the art.

Examples of molecular modelling systems are the CHARMm and QUANTA programs (Polygen Corporation, Waltham, Mass.). CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modelling of drugs interactive with specific proteins, such as Rotivinen, et al.) 1988, Acta Pharmaceutical Fennica 97: 159-166); Ripka (1988 New Scientist 54-57); McKinaly and Rossmann (1989, Annu. Rev. Pharmacol. Toxiciol. 29: 111-122); Perry and Davies, OSAR: Quantitative Structure-Activity Relationships in Drug Design pp. 189-193 Alan R. Liss, Inc. 1989; Lewis and Dean (1989, Proc. R. Soc. Lond. 236: 125-140 and 141-162); and, with respect to a model receptor for nucleic acid components, Askew, et al. (1989, J. Am. Chem. Soc. 111: 1082-1090). Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds which are inhibitors or activators.

Compounds identified via assays such as those described herein may be useful, for example, in elaborating the biological function of the MEX3C gene product, and for ameliorating body weight disorders. Assays for testing the efficacy of compounds identified in the cellular screen can be tested in animal model systems for body weight disorders. Such animal models may be used as test substrates for the identification of drugs, pharmaceuticals, therapies and interventions which may be effective in treating such disorders. For example, animal models may be exposed to a compound, suspected of exhibiting an ability to ameliorate body weight disorder symptoms, at a sufficient concentration and for a time sufficient to elicit such an amelioration of body weight disorder symptoms in the exposed animals. The response of the animals to the exposure may be monitored by assessing the reversal of disorders associated with body weight disorders such as obesity. With regard to intervention, any treatments which reverse any aspect of body weight disorder-like symptoms should be considered as candidates for human body weight disorder therapeutic intervention. Dosages of test agents may be determined by deriving dose-response curves as known in the art.

In an alternate embodiment, weight gain therapy can be designed to reduce the level of endogenous MEX3C gene expression, e.g., using antisense or ribozyme approaches to inhibit or prevent translation of MEX3C mRNA transcripts; triple helix approaches to inhibit transcription of the MEX3C gene; or targeted homologous recombination to inactivate or “knock out” the MEX3C gene or its endogenous promoter. Because the MEX3C gene is expressed in the brain, delivery techniques should be preferably designed to cross the blood-brain barrier (see PCT WO89/10134, which is incorporated by reference herein in its entirety). Alternatively, the antisense, ribozyme or DNA constructs described herein could be administered directly to the site containing the target cells.

Antisense approaches involve the design of oligonucleotides (either DNA or RNA) that are complementary to mRNA. The antisense oligonucleotides will bind to the complementary mRNA transcripts and prevent translation. Absolute complementarily, although preferred, is not required. A sequence “complementary” to a portion of an RNA, as referred to herein, means a sequence having sufficient complementarily to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarily and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

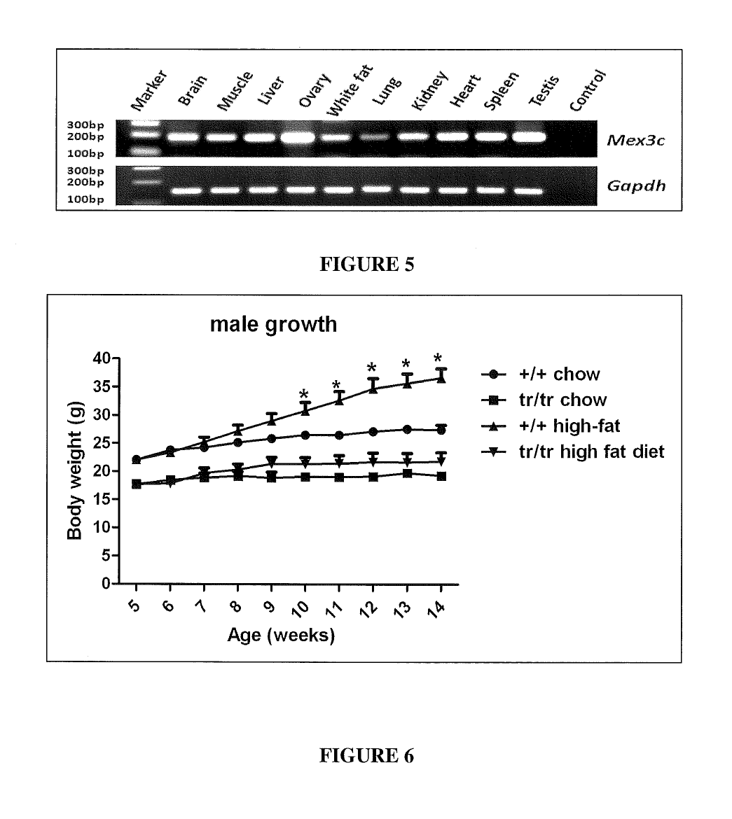

While antisense nucleotides complementary to the coding region sequence could be used, those complementary to the transcribed untranslated region are most preferred. Oligonucleotides that are complementary to the 5′ end of the message, e.g., the 5′ untranslated sequence up to and including the AUG initiation codon, should work most efficiently at inhibiting translation (see FIGS. 5A-5C). However, sequences complementary to the 3′ untranslated sequences of mRNAs have recently shown to be effective at inhibiting translation of mRNAs as well. See generally, Wagner, R., 1994, Nature 372: 333-335. Thus, oligonucleotides complementary to either the 5′- or 3′-non-translated, non-coding regions of MEX3C could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of MEX3C mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6: 958-976) or intercalating agents. (See, e.g., Zon, 1988, Pharm. Res. 5: 539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl)uracil, 5 -carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5 -methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an alpha-anomeric oligonucleotide. An .alpha.-anomeric oligonucleotide forms specific double-stranded hybrids with complementary RNA in which, contrary to the usual β-units, the strands run parallel to each other (Gautier et al., 1987, Nucl. Acids Res. 15: 6625-6641). The oligonucleotide is a 2′-O-methylribonucleotide (Inoue et al., 1987, Nucl. Acids Res. 15: 6131-6148), or a chimeric RNA-DNA analogue (Inoue et al., 1987, FEBS Lett. 215: 327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16: 3209), methylphosphonate oligonucleotides can be prepared by use of controlled pore glass polymer supports (Sarin et al., 1988, Proc. Natl. Acad. Sci. U.S.A. 85: 7448-7451), etc.

The antisense molecules should be delivered to cells which express the MEX3C in vivo, e.g., neural tissue. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous MC4-R transcripts and thereby prevent translation of the MC4-R mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296: 39-42), etc. Any type of plasmid, cosmid, YAC or viral vector can be used to prepare the recombinant DNA construct which can be introduced directly into the tissue site; e.g., the choroid plexus or hypothalamus. Alternatively, viral vectors can be used which selectively infect the desired tissue; (e.g., for brain, herpesvirus vectors may be used), in which case administration may be accomplished by another route (e.g., systemically).

Ribozyme molecules designed to catalytically cleave MEX3C mRNA transcripts can also be used to prevent translation of MEX3C mRNA and expression of MEX3C. (See, e.g., PCT International Publication WO90/11364, published Oct. 4, 1990; Sarver et al., 1990, Science 247: 1222-1225). While ribozymes that cleave mRNA at site specific recognition sequences can be used to destroy MEX3C mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334: 585-591. There are hundreds of potential hammerhead ribozyme cleavage sites within the nucleotide sequence of human MEX3C cDNA (see FIGS. 5A-5C). Preferably the ribozyme is engineered so that the cleavage recognition site is located near the 5′ end of the MEX3C mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224: 574-578; Zaug and Cech, 1986, Science, 231: 470-475; Zaug, et al., 1986, Nature, 324: 429-433; published International patent application No. WO 88/04300 by University Patents Inc.; Been and Cech, 1986, Cell, 47: 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in MEX3C.

As in the antisense approach, the ribozymes can be composed of modified oligonucleotides (e.g. for improved stability, targeting, etc.) and should be delivered to cells which express the MEX3C in vivo. A preferred method of delivery involves using a DNA construct “encoding” the ribozyme under the control of a strong constitutive pol III or pol II promoter, so that transfected cells will produce sufficient quantities of the ribozyme to destroy endogenous MEX3C messages and inhibit translation. Because ribozymes unlike antisense molecules, are catalytic, a lower intracellular concentration is required for efficiency.

Endogenous MEX3C gene expression can also be reduced by inactivating or “knocking out” the MEX3C gene or its promoter using targeted homologous recombination (e.g., see Smithies et al., 1985, Nature 317: 230-234; Thomas & Capecchi, 1987, Cell 51: 503-512; Thompson et al., 1989 Cell 5: 313-321; each of which is incorporated by reference herein in its entirety). For example, a mutant, non-functional MEX3C (or a completely unrelated DNA sequence) flanked by DNA homologous to the endogenous MEX3C gene can be used, with or without a selectable marker and/or a negative selectable marker, to transfect cells that express MEX3C in vivo. Insertion of the DNA construct, via targeted homologous recombination, results in inactivation of the MEX3C gene. Such approaches are particularly suited in the agricultural field where modifications to ES (embryonic stem) cells can be used to generate animal offspring with an inactive MEX3C (e.g., see Thomas & Capecchi 1987 and Thompson 1989, supra). However this approach can be adapted for use in humans provided the recombinant DNA constructs are directly administered or targeted to the required site in vivo using appropriate viral vectors, e.g., herpes virus vectors for delivery to brain tissue; e.g., the hypothalamus and/or choroid plexus.

Alternatively, endogenous MEX3C gene expression can be reduced by targeting deoxyribonucleotide sequences complementary to the regulatory region of the MEX3C gene (i.e., the MEX3C promoter and/or enhancers) to form triple helical structures that prevent transcription of the MEX3C gene in target cells in the body. (See generally, Helene, C. 1991, Anticancer Drug Des., 6 (6): 569-84; Helene, C., et al., 1992, Ann, N.Y. Acad. Sci., 660: 27-36; and Maher, L. J., 1992, Bioassays 14 (12): 807-15).

Agents useful in the methods as disclosed herein can also inhibit gene expression (i.e. suppress and/or repress the expression of the gene). Such agents are referred to in the art as “gene silencers” and are commonly known to those of ordinary skill in the art. Examples include, but are not limited to a nucleic acid sequence, for an RNA, DNA or nucleic acid analogue, and can be single or double stranded, and can be selected from a group comprising nucleic acid encoding a protein of interest, oligonucleotides, nucleic acids, nucleic acid analogues, for example but are not limited to peptide nucleic acid (PNA), pseudo-complementary PNA (pc-PNA), locked nucleic acids (LNA) and derivatives thereof etc. Nucleic acid agents also include, for example, but are not limited to nucleic acid sequences encoding proteins that act as transcriptional repressors, antisense molecules, ribozymes, small inhibitory nucleic acid sequences, for example but are not limited to RNAi, shRNAi, siRNA, micro RNAi (miRNA), antisense oligonucleotides, etc.

Examples of the foregoing include, but are not limited to:

(A) the following dsRNA agents, which are directed to the target segments described below:

| | | |
|---|---|---|
| Target sequence 1: | AAGAGGACGAGGAGGAGGGGG | (SEQ ID NO: 1) |
| Position in coding region: | 326 | |
| Sense strand siRNA: | GAGGACGAGGAGGAGGGGGUU | (SEQ ID NO: 2) |
| Antisense strand siRNA: | CCCCCUCCUCCUCGUCCUCUU | (SEQ ID NO: 3) |
| Target sequence 2: | AAGCGGAGCTGGACGGAGACC | (SEQ ID NO: 4) |
| Position in coding region: | 350 | |
| Sense strand siRNA: | GCGGAGCUGGACGGAGACCUU | (SEQ ID NO: 5) |
| Antisense strand siRNA: | GGUCUCCGUCCAGCUCCGCUU | (SEQ ID NO: 6) |
| Target sequence 3: | AAGCAGAGGAGGAGGACCGGT | (SEQ ID NO: 7) |
| Position in coding region: | 395 | |
| Sense strand siRNA: | GCAGAGGAGGAGGACCGGUUU | (SEQ ID NO: 8) |
| Antisense strand siRNA: | ACCGGUCCUCCUCCUCUGCUU | (SEQ ID NO: 9) |
| Target sequence 4: | AACGGGGAGCAGGCGGCCCTG | (SEQ ID NO: 10) |
| Position in coding region: | 652 | |
| Sense strand siRNA: | CGGGGAGCAGGCGGCCCUGUU | (SEQ ID NO: 11) |
| Antisense strand siRNA: | CAGGGCCGCCUGCUCCCCGUU | (SEQ ID NO: 12) |
| Target sequence 5: | AAAGAGCGTCAACACCACCGA | (SEQ ID NO: 13) |
| Position in coding region: | 681 | |
| Sense strand siRNA: | AGAGCGUCAACACCACCGAUU | (SEQ ID NO: 14) |
| Antisense strand siRNA: | UCGGUGGUGUUGACGCUCUUU | (SEQ ID NO: 15) |
| Target sequence 6: | AACACCACCGAGTGCGTCCCG | (SEQ ID NO: 16) |
| Position in coding region: | 691 | |
| Sense strand siRNA: | CACCACCGAGUGCGUCCCGUU | (SEQ ID NO: 17) |
| Antisense strand siRNA: | CGGGACGCACUCGGUGGUGUU | (SEQ ID NO: 18) |
| Target sequence 7: | AAAATTAAAGCACTGAGAGCC | (SEQ ID NO: 19) |
| Position in coding region: | 760 | |
| Sense strand siRNA: | AAUUAAAGCACUGAGAGCCUU | (SEQ ID NO: 20) |
| Antisense strand siRNA: | GGCUCUCAGUGCUUUAAUUUU | (SEQ ID NO: 21) |
| Target sequence 8: | AATTAAAGCACTGAGAGCCAA | (SEQ ID NO: 22) |
| Position in coding region: | 762 | |
| Sense strand siRNA: | UUAAAGCACUGAGAGCCAAUU | (SEQ ID NO: 23) |
| Antisense strand siRNA: | UUGGCUCUCAGUGCUUUAAUU | (SEQ ID NO: 24) |
| Target sequence 9: | AAAGCACTGAGAGCCAAGACA | (SEQ ID NO: 25) |
| Position in coding region: | 766 | |
| Sense strand siRNA: | AGCACUGAGAGCCAAGACAUU | (SEQ ID NO: 26) |
| Antisense strand siRNA: | UGUCUUGGCUCUCAGUGCUUU | (SEQ ID NO: 27) |
| Target sequence 10: | AAGACAAACACGTATATCAAG | (SEQ ID NO: 28) |
| Position in coding region: | 781 | |
| Sense strand siRNA: | GACAAACACGUAUAUCAAGUU | (SEQ ID NO: 29) |
| Antisense strand siRNA: | CUUGAUAUACGUGUUUGUCUU | (SEQ ID NO: 30) |
| Target sequence 11: | AAACACGTATATCAAGACTCC | (SEQ ID NO: 31) |
| Position in coding region: | 786 | |
| Sense strand siRNA: | ACACGUAUAUCAAGACUCCUU | (SEQ ID NO: 32) |
| Antisense strand siRNA: | GGAGUCUUGAUAUACGUGUUU | (SEQ ID NO: 33) |
| Target sequence 12: | AAGACTCCTGTTCGTGGTGAA | (SEQ ID NO: 34) |
| Position in coding region: | 799 | |
| Sense strand siRNA: | GACUCCUGUUCGUGGUGAAUU | (SEQ ID NO: 35) |
| Antisense strand siRNA: | UUCACCACGAACAGGAGUCUU | (SEQ ID NO: 36) |
| Target sequence 13: | AAGAGCCCATTTTTGTTGTCA | (SEQ ID NO: 37) |
| Position in coding region: | 818 | |
| Sense strand siRNA: | GAGCCCAUUUUUGUUGUCAUU | (SEQ ID NO: 38) |
| Antisense strand siRNA: | UGACAACAAAAAUGGGCUCUU | (SEQ ID NO: 39) |
| Target sequence 14: | AAGGAAAGAAGATGTTGCCAT | (SEQ ID NO: 40) |
| Position in coding region: | 843 | |
| Sense strand siRNA: | GGAAAGAAGAUGUUGCCAUUU | (SEQ ID NO: 41) |
| Antisense strand siRNA: | AUGGCAACAUCUUCUUUCCUU | (SEQ ID NO: 42) |
| Target sequence 15: | AAAGAAGATGTTGCCATGGCC | (SEQ ID NO: 43) |
| Position in coding region: | 847 | |
| Sense strand siRNA: | AGAAGAUGUUGCCAUGGCCUU | (SEQ ID NO: 44) |
| Antisense strand siRNA: | GGCCAUGGCAACAUCUUCUUU | (SEQ ID NO: 45) |
| Target sequence 16: | AAGATGTTGCCATGGCCAAAA | (SEQ ID NO: 46) |
| Position in coding region: | 851 | |
| Sense strand siRNA: | GAUGUUGCCAUGGCCAAAAUU | (SEQ ID NO: 47) |
| Antisense strand siRNA: | UUUUGGCCAUGGCAACAUCUU | (SEQ ID NO: 48) |
| Target sequence 17: | AAAAGAGAGATCCTCTCAGCT | (SEQ ID NO: 49) |
| Position in coding region: | 868 | |
| Sense strand siRNA: | AAGAGAGAUCCUCUCAGCUUU | (SEQ ID NO: 50) |
| Antisense strand siRNA: | AGCUGAGAGGAUCUCUCUUUU | (SEQ ID NO: 51) |
| Target sequence 18: | AAGAGAGATCCTCTCAGCTGC | (SEQ ID NO: 52) |
| Position in coding region: | 870 | |
| Sense strand siRNA: | GAGAGAUCCUCUCAGCUGCUU | (SEQ ID NO: 53) |
| Antisense strand siRNA: | GCAGCUGAGAGGAUCUCUCUU | (SEQ ID NO: 54) |
| Target sequence 19: | AAACAAAAATGGGCCTGCCCT | (SEQ ID NO: 55) |
| Position in coding region: | 921 | |
| Sense strand siRNA: | ACAAAAAUGGGCCUGCCCUUU | (SEQ ID NO: 56) |
| Antisense strand siRNA: | AGGGCAGGCCCAUUUUUGUUU | (SEQ ID NO: 57) |
| Target sequence 20: | AAAAATGGGCCTGCCCTGGGA | (SEQ ID NO: 58) |
| Position in coding region: | 925 | |
| Sense strand siRNA: | AAAUGGGCCUGCCCUGGGAUU | (SEQ ID NO: 59) |
| Antisense strand siRNA: | UCCCAGGGCAGGCCCAUUUUU; | (SEQ ID NO: 60) |

(B) RNAi agents of (A) above with 1, 2, 3, or 4 consecutive nucleotides deleted from the 5′ terminus, the 3′ terminus, or both the 5′ and 3′ terminus, of either, or both, strands thereof; and

(C) RNAi agents of (A) or (B) above with 1, 2, 3, 4, 5, 6, 7 or 8 consecutive nucleotides added to the the 5′ terminus, the 3′ terminus, or both the 5′ and 3′ terminus, of either, or both, strands thereof.

Numerous other variations of the foregoing will be appreciated by those skilled in the art and the foregoing are to be construed as including dsRNA agents having additional compounds coupled thereto, modified backbones, etc.

As used herein, agents useful in the method as inhibitors of MEX3c expression and/or inhibition of MEX3c protein function can be any type of entity, for example but are not limited to chemicals, nucleic acid sequences, nucleic acid analogues, proteins, peptides or fragments thereof. In some embodiments, the agent is any chemical, entity or moiety, including without limitation, synthetic and naturally-occurring non-proteinaceous entities. In certain embodiments the agent is a small molecule having a chemical moiety.

In alternative embodiments, agents useful in the methods as disclosed herein are proteins and/or peptides or fragment thereof, which inhibit the gene expression of MEX3c or the function of the MEX3c protein. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof.

Alternatively, agents useful in the methods as disclosed herein as inhibitors of MEX3c can be a chemicals, small molecule, large molecule or entity or moiety, including without limitation synthetic and naturally-occurring non-proteinaceous entities. In certain embodiments the agent is a small molecule having the chemical moieties as disclosed herein.

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alfa, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an activ e compound(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Of course, the liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

Subjects to be treated include subjects in which inhibiting fat deposition is desired. Subjects to be treated include, but are not limited to, subjects afflicted with obesity, diabetes (including type II diabetes), syndrome X, glaucoma, hyperlipidemia, hyperglycemia and hyperinsulinemia, vascular conditions associated with elevated sterol and/or stanol levels, arteriosclerotic disease, renal disease, hepatic disease, metabolic syndrome, etc.

The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.01 or 0.1 to about 50 or 100 mg/kg will have therapeutic efficacy, with all weights being calculated based upon the weight of the active compound, including the cases where a salt is employed.

The present invention is explained in greater detail in the following non-limiting Examples.

### Experimental

### A. Materials and Methods

Generation of Mex3c gene trap mouse. The Mex3c gene trap ES cell line (allele symbol: Mex3c^{Gt(DD0642)Wtsi}, MGI:3764947) was obtained from the Sanger Institute Gene Trap Resource (SIGTR) (line DD0642). The ES cells were microinjected into mouse blastocysts and the resulted chimera males were mated with C57/BL6 females to obtain heterozygous Mex3c gene trap mice. The heterozygous mice were mated with FVB/N mice and the resulting male and female heterozygotes were intercrossed to obtain homozygous gene trap mice in the mixed FVB/129/C57/BL6 background. Mice were housed in the animal facility of Wake Forest University Health Sciences. Experiments were conducted in accordance with the National Research Council publication Guide for Care and Use of Laboratory Animals.

Genotyping. Ear biopsies were used for genotyping as described (Agoulnik et al., 2002). The gene trap allele was detected by a positive PCR amplification of a 419 by product with primer TrapF (ttcaacatcagccgctacag) and TrapR (ctcgtcctgcagttcattca). Homozygous gene trap mice were distinguished from heterozygous mice by PCR with primer pair D18Mit210 (tgggcagaagtataactaaatcca and ttcaaaccgtatgcctttcc). A 122 by PCR product will be obtained from the un-trapped C57/BL6 and FVB allele and a 146 by PCR product obtained from the trapped 129/Sv allele. The size difference was resolved on a 3% agarose gel stained with ethidium bromide.

MEX3C antibody, SDS-PAGE and Western blotting analysis. Rabbit anti-MEX3C antibody was generated using a KLH-conjugated peptide corresponding to amino acids 330-348 of human MEX3C NP_{—}057710 (DPSGNMKTQRRGSQPSTPRC). Antibody was affinity-purified with peptide coupled agarose as described previously (Zhao et al., 2005). The specificity of the antibody was confirmed by different methods. siRNA specific to human MEX3C mRNA (5′-CCUAGCAGUUGACUCUCCUGCCUUU-3′) was purchased from Invitrogen (RKHD2HSS122041), control siRNA with similar GC content to MEX3C SiRNA was from Santa Cruz (SC-36869). SDS-PAGE and Western blotting assays were carried out as described previously (Lu et al., 2008; Sambrook, 1989). Horseradish peroxidase (HRP)-conjugated secondary antibodies were purchased from Pierce. Chemiluminescent reagents from Pierce were used to visualize the protein signals under the LAS-3000 system from Fujifilm.

Immunostaining and β-gal staining. Isolated sperm were washed with PBS, fixed with 4% PFA at 4° C. for 30 min and permeablized with 0.15% Triton X-100 at room temperature for 30 min. The sperm were washed with PBS and incubated at RT with 10% goat serum for 1 h. Then the sperm were incubated at RT with 3% goat serum diluted anti-MEX3C (1:100) or pre-immune IgG (1:100, as a negative control) for 1 h. Following washing with PBST, the sperm were incubated with Alexa Fluor 594 conjugated anti-rabbit antibody (Invitrogen). After washing the sperm were applied to glass slide for observation.

Cryosections were used for immunostaining. To detect the expression of MEX3C protein, sections were incubated at RT with 3% goat serum diluted anti-MEX3C (1:100) or pre-immune IgG (1:100, as a negative control) for 1 h after blocking with 5% goat serum. After the sequential incubation with biotin conjugated anti-rabbit secondary antibody (Vector) and HRP-conjugated streptoavidin, the sections were incubated with DAB to visualize the expression of MEX3C. For double immunostaining of β-gal and α-actin, cross sections were prepared from cryopreserved blood vessel, uterus, oviduct, intestine and ureter. The sections were blocked at RT for 1 h with 10% goat serum, incubated sequentially with rabbit anti-β-gal antibody (1:500, Abcam), FITC-conjugated donkey anti-rabbit secondary antibody (Jackson laboratories, 1:100), mouse anti-α-actin antibody (1:200, Chemicon) and Texas red conjugated anti-mouse secondary antibody (Jackson laboratories, 1:100). The nuclei were stained by DAPI.

β-gal staining was performed as described (Beddington et al., 1989).

Images were acquired with an Axio M1 microscope equipped with an AxioCam MRc digital camera (Carl Zeiss). Different images were assembled into one file with Adobe Photoshop, with necessary resizing, rotation and cropping.

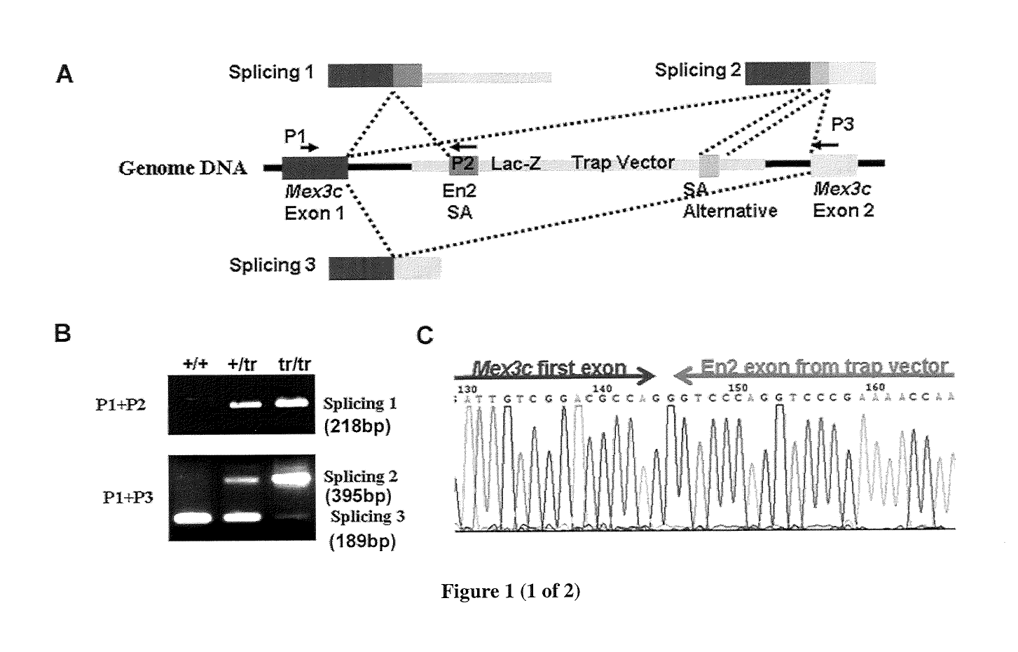

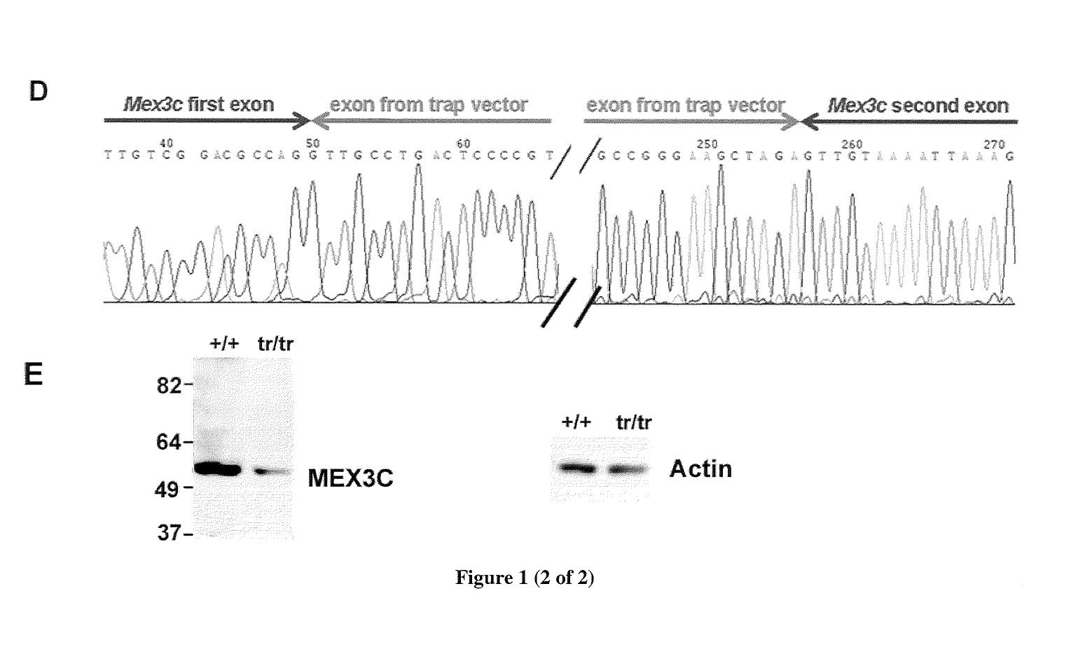

RT-PCR. Total RNA was extracted from mouse tissues with an RNeasy Protect Mini Kit (Qiagen). RNA samples were treated with Turbo DNase (Ambion) to eliminate DNA, and were then reverse transcribed with random primers by Moloney Murine Leukemia Virus reverse transcriptase (lnvitrogen). Primer Mex3cF (5′gaaggaagagcgtcaacacc3′, (SEQ ID NO: 61) listed as P1 in FIG. 1) and Mex3cR (5′gccatggcaacatcttcttt3′, (SEQ ID NO: 62) listed as P3 in FIG. 1) were used to detect normal Mex3c mRNA. Mex3cF and TrapV (5′gccagggttttcccagtcacgac3′ (SEQ ID NO: 63) listed as P2 in FIG. 1) was used to detect transcripts containing the Lac-Z cDNA. The thermal cycle parameters were: 94° C. 4 min, followed by 35 cycles of 94° C. 30 sec, 58° C. 30 sec, 72° C. 30 sec.

Food consumption. Normal control and homozygous gene trap mice (2˜3 month) in the mixed background were used in the experiment. Five animals of each sex and genotype were housed in cage for at least 4 days before the assay. Purina rodent chow and water were available ad libitum. Consumption of food was measured for each group every 2 days for a successive 10 days. Body weight of the animals was measured on the first and last day of the assay. The food consumption was expressed as gram/day/100 g body weight.

Blood glucose, triglyceride and cholesterol assay. Blood glucose was assayed from tail whole blood by Roche ACCU-CHEK Active and Test Strips. Glucose was also assayed from serum by Beckman Synchron Clinical System CX5CE. Serum cholesterol concentration was assayed by Beckman Synchron Clinical System CX5CE. Assay reagents were from Carolina Liquid Chemistries. Triglyceride concentration was determined with the Triglyceride Assay kit from Cayman Chemical (Ann Arbor, Mich.).

Vascular-stromal fraction (VSF) cell isolation and differentiation. VSF cells from gonadal WAT were isolated from adult mice as described (Hauner et al., 1987). Adipogenic, osteogenic and chondrogenic differentiation were performed as described before (Zuk et al., 2001). Briefly, cells were plated at about 1×10⁴ cells/cm² and cultured in growth medium (MEM-α with 10% FBS, antibiotics) until they reached 100% confluency, when adipogenic or osteogenic differentiation medium was replaced. Adipogenic differentiation medium included dexamethasone (1 μM), 3-isobutyl-1-methylxanthine (500 μM), Indomethacin (100 μM) and insulin (10 μg/ml) in growth medium. Osteogenic differentiation medium was purchased from Lonza (PT-4120) which included dexamethasone (0.1 μM), ascorbic acid 2-phosphate (200 μM) and glycerol 2-phosphate (10 mM). Medium was replaced every 3 days. For adipogenic differentiation, cells were stained for lipid vacuoles by oil red O 2 weeks after induction. For osteogenic differentiation, cells were stained for calcium deposits by Alizarin Red S 4 weeks after induction.

Fecal lipid content. Fecal lipids were extracted as described by Folch et al (Folch et al., 1957). Briefly, feces were collected from mice housed individually in metabolic cages over a 24-h period. One-hundred-milligram aliquots of feces were cleaned and dried for 1 h at 70° C., incubated with 2 ml of chloroform-methanol (2:1) for 30 min at 60° C. with constant agitation, and then centrifuged. Water (1 ml) was added to the supernatant, and following vortexing, phase separation was induced by low-speed centrifugation (2,000 rpm for 10 min). The lower chloroform phase was then removed and transferred to a new tube, and the sample was evaporated to dryness. The mass of the total lipid was obtained by subtracting the weight of the tube with lipid by the weight of the same tube without lipid.

Results

1. Generation of Mex3c Gene Trap Mouse.

We have been using transgenic insertional mutagenesis strategies to generate animal models for the study of human diseases (Lu et al., 2007). One of the transgenic lines showing impaired fertility was found to harbor three transgene insertions, two of which in regions without known genes and one in Mex3c gene whose function is unknown (data not shown). To exclude the possible influence of the two insertions in regions without genes, we generated a Mex3c mutation mouse line through injection of Mex3c gene trap ES cell line (clone dd0642, The Sanger Institute Gene Trap Resource), in which Mex3c gene was mutated by the integration of the gene trap vector pGT1xrT2 into the only intron of Mex3c (FIG. 1A). This mutated allele was designated as Mex3c^{Gt(DD0642)Wtsi}. RT-PCR analysis revealed three types of transcripts from the mutated allele (FIG. 1B): Transcript 1 contains the first exon of Mex3c and the Lac-Z cDNA from the gene trap vector, the protein product of which contains the N-terminal 56 amino acids of MEX3C and full length β-galactodase (β-gal) (FIG. 1B, C). Transcript 2 contains an extra exon from the gene trap vector between Mex3c exon 1 and exon 2 (FIG. 1B, D). Since there are numerous in frame stop codons within the inserted exon, full-length MEX3C protein can't be generated from this transcript. Although an internal ATG codon in Mex3c exon 2 with consensus Kozak sequence could possibly generate a protein of 372 amino acids containing the second KH domain and the C-terminal zinc finger domain, this protein species was not detected in tissue from the homozygous mice (FIG. 1E). Transcript 3 corresponds to intact Mex3c mRNA, which amounted to 5-10% of normal control mice and will give rise to full-length MEX3C protein.

We examined the protein expression of MEX3C in Mex3c^{Gt(DD0642)Wtsi}/Mex3c^{Gt(DD0642)Wtsi} mice in the testis, one of the organs normally found to highly express Mex3c mRNA (see below). As shown in FIG. 1E, the antibody detected a strong band in the control lysate and a weak band in the mutant lysate at the size of about 58 kDa. We believe that these bands are specific MEX3C bands since the specificity of MEX3C antibody has been confirmed by multiple methods, including specific reaction with bacteria expressed GST-MEX3C fusion protein and 293T expressed Flag-MEX3C fusion protein (data not shown). The Immunostaining with MEX3C antibody to be described below also confirmed the specificity of the antibody. Although NCBI database predicts mouse MEX3C protein to be 652 AA, this 58 kDa species could be the result of partial protein degradation or a testis-specific version resulted from alternative splicing. We noticed that all the available full-length cDNA records in public databases encode a MEX3C protein of 464 AA, while there is no single full-length cDNA record encoding a MEX3C protein of 652 AA. Thus it could also be possible that in mouse the 464 AA MEX3C species is the major form and the 58 kDa protein is the result of posttranslational modification of the 464 AA MEX3C protein. Anyway, the data suggested that homozygous mutant mice expressed MEX3C protein at a low level, consistent with the finding that intact Mex3c mRNA is present in homozygous mutant mice only at low levels. Thus RNA and protein data both showed that this Mex3c^{Gt(DD0642)Wtsi} allele was not a null allele but a hypomorphic one.

### 2. Expression Profile of Mex3c Gene

To examine the expression profile of Mex3c mRNA, we extracted RNA from various mouse tissues and performed RT-PCR. We found that Mex3c mRNA was expressed in all the tissues examined although the expression varied among tissues, with higher expression in the testis and the ovary (data not shown). Since the Lac-Z mRNA in the trapped allele (transcript 1 in FIG. 1) is under the control of the Mex3c promoter enabling it to trace the normal tissue expression of Mex3c mRNA, we stained tissues from Mex3c^{Gt(DD0642)Wtsi}/+ mice with X-Gal to gain an idea about which tissues or cell types might have higher expression of Mex3c mRNA.

Strong positive staining was observed in the ovary and the testis, which is consistent with the RT-PCR data (not shown). The brain and ductal structures, such as the urinal tract, the blood vessel, the uterus, the oviduct and the intestine were also strongly positive (data not shown). The liver, kidney, lung, spleen, skeletal muscle or white fat tissues did not stain positive in this assay although they were positive in RT-PCR assay (not shown). It therefore appears that Mex3c is widely expressed in various tissues with higher expression levels in the β-gal positive tissues such as the testis, ovary and the brain.

Examination of tissue sections from the testis showed that β-gal positive cells could be found along the basement of the seminiferous tubules (not shown), which could be spermatogenic cells at their early differentiation stage. Late spermatids, judged by their central localization in the seminiferous tubules, were also positive (not shown). In the ovary, strong staining was observed in granulosa cells, while the oocytes, the corpus lutea and other interstitial cells in the ovary are β-gal negative (not shown). Thus Mex3c has higher expression in early and later stages of spermatogenic cells in the testis, and higher expression in the granulosa cells in the ovary.

In the cortex of the brain, cells in layers II, III and beyond were positively stained (data not shown). In the hippocampus, neurons in CA1-3 and dentate gyrus stained positive, with neurons in CA1 being strongly positive (data not shown). In the cerebellum, Purkinje cells were stained positive (data not shown). When the sections of the uterus (data not shown), the oviduct (data not shown), the ureter (data not shown) and the aorta (B) were stained, the localization of the β-gal positive cells suggested they were expressed in smooth muscle cells. To confirm this, we co-immunostained sections from the uterus, oviduct, ureter, aorta and intestine with antibodies against β-gal and α-actin, a marker for smooth muscle cells, and found that the β-gal positive cells were indeed exclusively α-actin positive (data not shown for oviduct section, similar data for the uterus, the intestine, the ureter and the blood vessel not shown), which indicated that in these ductal structures smooth muscle cells showed high Mex3c expression.

To examine whether β-gal staining reflected the expression of MEX3C protein expression, we immunostained the brain sections with MEX3C antibody. A comparison of the β-gal staining and MEX3C immunostaining of the cortex, the hippocampus and the cerebella revealed that they have similar staining pattern (data not shown). In both staining, the cells in layer II and III of the cerebra, the neurons in CA1-3, and the Purkinje cells were positively stained. These data on one hand indicated that the β-gal staining reflected the expression of MEX3C protein, on the other hand further confirmed the specificity of our MEX3C antibody.

In summary, our data showed that Mex3c was highly expressed in neuronal cells of the nerve system, granulosa cells in the ovary, early spermatogenic cells and late haploid spermatids in the testis, and smooth muscle cells in various organs. There is a low level of Mex3c mRNA in the other cell types.

### 3. Significantly Reduced Adiposity in Adult MEX3C Deficient Mice

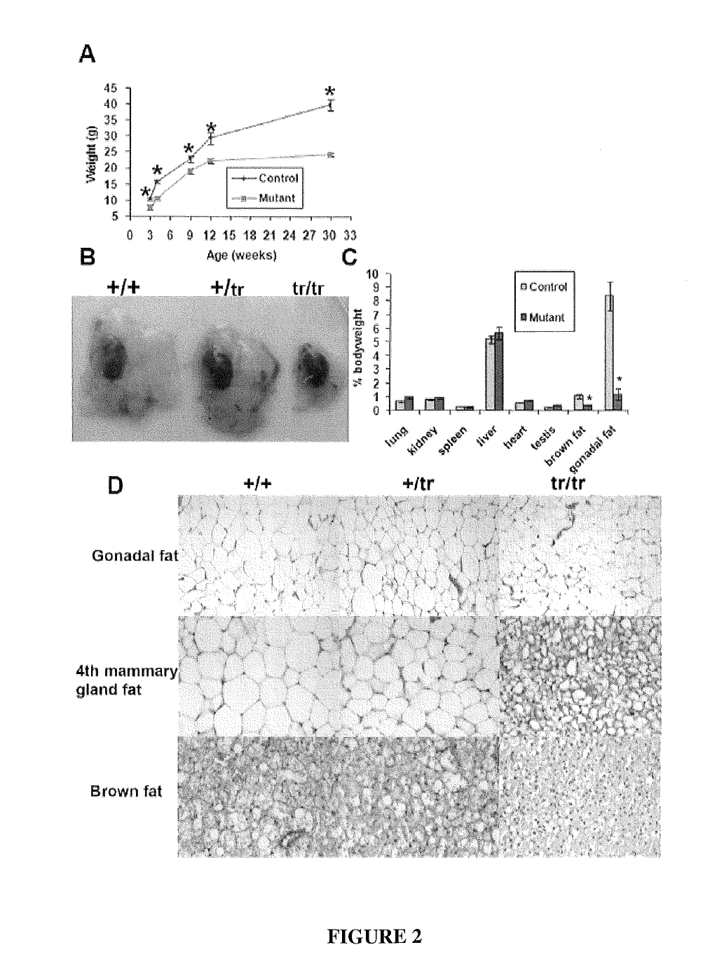

MEX3C deficiency did not seem to affect embryonic development since the mutants were apparently born normal and genotypes of pups from +/tr×+/tr matings followed the Mendel ratio. The mutant mice were viable and fertile, except that they were slightly growth retarded during development (FIG. 2A). Their body length was about 85%-90% of that of controls while the bodyweight of both sexes was significantly reduced compared with age-matched controls, which is especially dramatic with the increase of age (FIG. 2A). It is noteworthy that tr/tr mice gain little weight after the age of 3 months. When the mice were sacrificed and the internal organs examined, we observed a significantly reduced fat deposition in tr/tr mice. Smaller fat pads were observed in all white fat tissues including gonadal fat (FIG. 2B, 2D), perirenal fat (FIG. 2C), visceral fat, mammary gland fat and subcutaneous fat (data not shown). Although with significant individual variation, mutants have 1/10 to ¼ gonadal fats of age-matched controls after controlled by body weight. Brown fat was also reduced in mutants to a less degree (FIG. 2D). Interestingly, the size of other internal organs expressed as percentage bodyweight was not reduced in the mutants (FIG. 2C, 2D).

Decreased fat mass can result from decreased adipocyte numbers (hypoplasia) or decreased adipocyte size (hypotrophy)³⁸. We examined different fat tissues and found that adipocyte volume of mutant gonadal fat was only slightly smaller than that of controls. Because of the huge difference in gonadal fat mass between mutants and controls, we concluded that both hypoplasia and hypotrophy underlay the reduced gonadal fat mass (FIG. 2E). For mammary gland fat pads and brown adipose tissue, adipocytes were clearly smaller and less differentiated, with less lipid storage (FIG. 2E).

### 4. Energy Intake of Mutant Mice

Although MEX3C-deficient mice consumed significantly less food than +/+ mice as absolute amount of daily food eaten, they did not show a significant difference if daily food consumption was controlled by body weight (Table 1). Although fat tissue contributes relatively little to the total energy expenditure of an organism compared with lean mass³⁹, the use of total body weight to control food intake was suitable in this case since this was assayed in animals of 7-11 weeks, when fat mass is low even in control mice. Thus, reduced adiposity in tr/tr mice is unlikely the result of less food intake. To check whether MEX3C-deficient mice have defects in nutrition absorption, we compared the residual lipid content in the feces of control and tr/tr mice. Lipid content in the feces was not higher in the mutants; on the contrary, it was statistically lower than that of normal mice, although the difference was marginal (Table 1). The mass of daily feces between +/+, +/tr and tr/tr groups was not significantly different whether the raw feces mass or the ratio of feces/food consumed was compared (Table 1). Thus the nutrition absorption in tr/tr mice was normal and these data suggest that tr/tr mice were unlikely to have reduced energy intake.

TABLE 1
Food intake and feces profiles of age-matched mutants and controls

| | +/+ | +/tr | tr/tr | P value |
|---|---|---|---|---|
| | (mean ± SEM) | (mean ± SEM) | (mean ± SEM) | (+/+ ~tr/tr) |
| Raw food (g) | 4.52 ± 0.46 | 4.583 ± 0.1740 | 2.88 ± 0.22 | <0.05* |
| Food/body | 0.194 ± 0.018 | 0.1697 ± 0.012 | 0.175 ± 0.016 | n.s |
| Residual Feces lipid | 0.0358 ± 0.0010 | 0.0343 ± 0.0013 | 0.0302 ± 0.0016 | <0.05* |
| (% feces) | | | | |
| Daily feces mass (g) | 0.91 ± 0.06951 | 0.998 ± 0.06432 | 0.8450 ± 0.06465 | n.s |
| Feces/food consumed | 0.1973 ± 0.007459 | 0.2173 ± 0.008633 | 0.2113 ± 0.01232 | n.s |

At least 6 animals were included in each group. Apart from feces lipid data which were from both males and females, the other data were from males only. Food intake was performed on mice of 7-11 weeks. Remaining assays were performed on mice 10~20 weeks of age. One-way ANOVA analysis was performed followed by Turkey's Multiple Comparison Test.
*indicates significant difference between +/+ and tr/tr groups;
n.s. indicates not significant.

### 5. Glucose Metabolism and Insulin Sensitivity

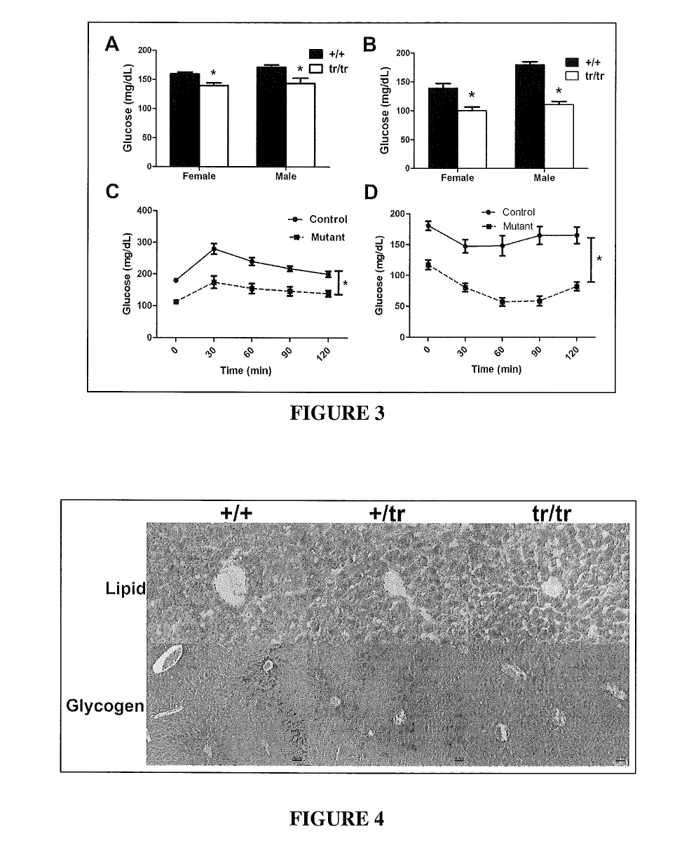

Adiposity can have a substantial impact on systemic glucose homeostasis^{40,41}. We found that MEX3C deficiency had a hypoglycemic effect, which was evident on non-fasting and fasting blood glucose (FIG. 3A, B). To investigate glucose homeostasis further, we performed intraperitoneal glucose and insulin tolerance tests. tr/tr mice showed improved maintenance of blood glucose levels upon bolus injection of glucose (FIG. 3C). In addition, they were very sensitive to bolus injection of insulin (FIG. 3D). These data demonstrated that MEX3C caused reduced adiposity and resulted in improved blood glucose maintenance and insulin sensitivity.

6. Reduced Liver Lipid Storage in tr/tr Mice

Our preliminary analysis of blood triglyceride and cholesterol content in a subset of mice revealed lower lipid content in tr/tr mice than in control mice (n=4 for each group, data not shown). Although preliminary, the data suggested that tr/tr mice were unlikely to have higher blood lipid content. We examined the content of triglyceride in the livers of tr/tr mice by Oil Red O staining of the liver cryosections. tr/tr mice had significantly less triglyceride content in their livers (FIG. 4A, red dot), demonstrating no ectopic energy storage. We consistently noted that +/tr mice showed intermediate liver lipid content between +/+ and tr/tr mice, indicating a dose-dependent effect of MEX3C on liver lipid content.

We also examined glycogen content in the livers using periodic acid Schiff staining; tr/tr mice had less glycogen storage in their livers than control mice, consistent with lower blood glucose levels in tr/tr mice (FIG. 4B, red). The data suggested that MEX3C deficiency helped the animals to avoid a positive energy balance.

### 7. MEX3C Deficiency Changed the Expression of Key Genes Involved in Glucose and Lipid Metabolism

MEX3C deficiency caused reduced adiposity but did not result in metabolic syndromes associated with lipodistrophy. MEX3C is an RNA-binding protein and its homologs MEX3B and MEX3D have been reported to regulate RNA stability^{35,36}. To gain a possible mechanistic insight into MEX3C deficiency, we extracted RNA from gonadal fat of 3 tr/tr mice and 3 +/+ mice, and compared gene expression profiles using Illumina DNA chips. Between the two groups, 111 genes met the criteria of having an expression difference of >1.5 fold and statistic significance of p<0.05. Among these genes, 102 genes were down regulated and only 9 are up regulated in the mutants. This indicated that to most possible RNA targets of MEX3C, MEX3C functioned to increase their stability. The expression of three genes known to play important roles in glucose and lipid metabolism changed significantly in tr/tr mice: Acaa2 (3.56 fold increased), Chrebp (official name Mlxipl, 1.63 fold increased for one probe, 1.52 fold increased for another probe), S14 (official name Thrsp, 1.67 fold increased). These were subsequently confirmed by quantitative RT-PCR (data not shown).

Acaa2 encodes acetyl-coenzyme A acyltransferase 2, also called mitochondrial 3-oxoacyl-coenzyme A thiolase, the rate-limiting enzyme in fatty acid β-oxidation⁴². Although two distinct β-oxidation systems exist in mammalian cells, mitochondrial⁴³ and peroxisomal⁴⁴, mitochondrial β-oxidation provides acetyl groups that can be degraded to CO₂ and H₂O to produce ATP, and is tightly coupled to the mitochondrial respiratory chain⁴⁵. Two mitochondrial thiolase activities are needed for β-oxidation of free fatty acids (FFA): the membrane-located β subunit of trifunctional protein (encoded by Hadhb) responsible for long chain fatty acid oxidation⁴⁶, and the matrix-located ACAA2 for medium and short chain fatty acid oxidation⁴⁷. No information on Acaa2 knockout or over-expression is available, while increased expression of ACAA2 is expected to increase the oxidation of FFA. Increasing FFA oxidation by knocking out of Acc2^{12,48} or knocking down of both Acc1 and Acc2⁴⁹ decreases blood FFA concentrations and inhibits lipogenesis. Thus, the gene expression analysis data are consistent with our observation that MEX3C-deficient mice have reduced adiposity and liver lipid content.

ChREBP is a glucose-induced transcription factor which is the insulin-independent mediator of glucose-induced lipogenesis^{50,51}. ChREBP-deficient mice display larger, glycogen-laden livers, smaller adipose depots, and decreased plasma FFA levels when consuming a standard rodent diet, suggesting a major role for ChREBP in utilizing circulating glucose to produce fatty acids⁵². In humans, CHREBP is associated with lipid concentrations and risk of coronary artery disease^{53,54}. Although CHREBP activity is controlled by phosphorylation⁵⁵, its mRNA level is regulated when glucose levels change^{56,57}. S14 gene is a downstream target of ChREBP responding to glucose^{58,59}. Initial work reported that knockout of S14 gene impaired lipogenesis in the mammary gland but not in the liver^{60,61} and it was subsequently found that this was possibly due to the compensation of S14r, a gene with overlapping function⁵⁸. Our observation of improved maintenance of glucose in MEX3C deficient mice is consistent with the finding of elevated expression of ChREBP and S14 genes. Increased ACAA2 expression in MEX3C deficient mice could decrease the plasma FFA concentration which resulted in reduced adiposity in spite of the elevated expression of ChREBP and S14.

### 8. Wide Expression of Mex3c Gene in Mouse Tissues

The fact that MEX3C deficiency leads to slight growth retardation indicates that MEX3C deficiency affects multiple tissues. To further examine this aspect, we examined the expression of Mex3c in various tissues by RT-PCR. Mex3c was expressed in all the tissues examined, although to varying degrees (FIG. 5). The wide expression of Mex3c in various tissues suggests that multiple tissues could have been affected by MEX3C deficiency to obtain the observed phenotype in MEX3C deficient mice.

### 9. MEX3C Deficient Mice are Resistant to Diet-Induced Obesity

To examine whether MEX3C deficiency will protect mice from high-fat diet induced obesity, we fed control and MEX3C deficient mice with a high-fat diet (Harlan, TD.08811, containing 17.3% protein, 47.6% carbohydrate and 23.2% fat, which contributing 14.8%, 40.6% and 44.6% calories respectively, 4.7 kcal/g) from the age of 5 weeks for 9 weeks and monitored the growth of the mice weekly. As shown in FIG. 6, starting from 5 weeks on high-fat diet, normal control mice showed significantly larger body weight than those on chow diet (Prolab, RMH3000, with 22.5% protein, 52% carbohydrate and 5.4% fat contributing 26%, 60% and 14% calorie respectively; total calorie 3.5 Kcal/g); while MEX3C deficient mice showed no difference in body weight compared with MEX3C deficient mice on chow diet.

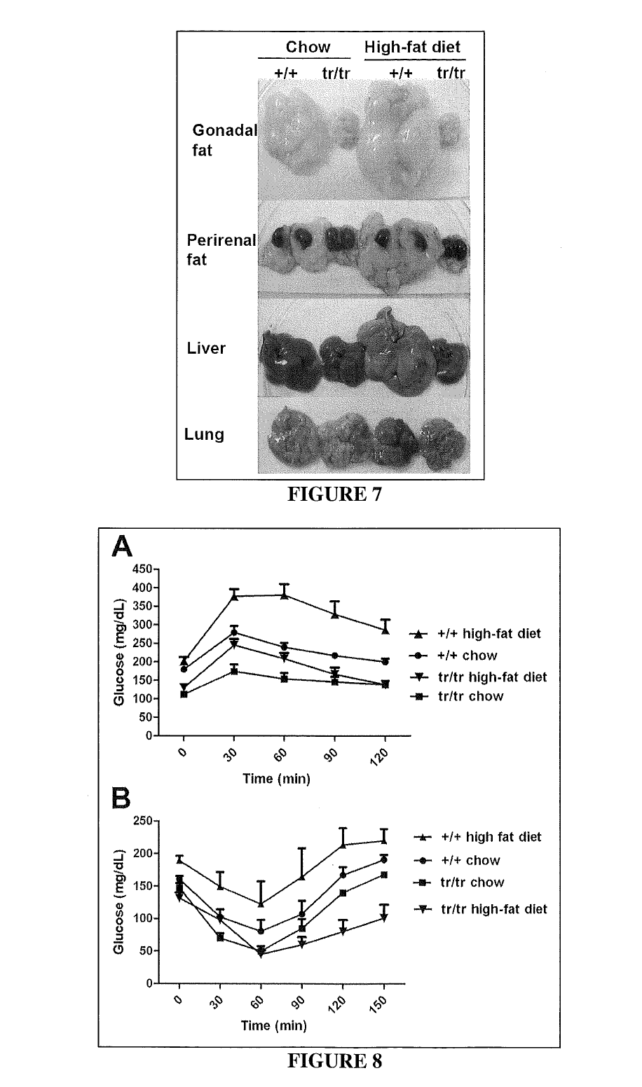

We compared the fat deposition of normal control and MEX3C deficient mice on normal and high-fat diet. While normal control mice on high-fat diet accumulated significantly more fat than mice on chow diet, exemplified by gonadal and perirenal fat in FIG. 7; MEX3C deficient mice did not show more fat deposition on high-fat diet. Normal control mice on high-fat diet clearly developed hepatomegaly and steatosis. This was not observed in MEX3C deficient mice on high-fat diet. The kidney and the liver in MEX3C deficient mice did not show size difference on either diet.

We examined the glucose maintenance in control and MEX3C deficient mice on high-fat diet. While normal control mice developed glucose intolerance and insulin insensitivity after 12 weeks on high-fat diet; MEX3C deficient mice showed much better glucose maintenance and insulin sensitivity than normal control mice on high-fat diet (FIG. 8). Thus MEX3C deficiency protected mice from diet-induced glucose and insulin intolerance.

### Conclusion

The data demonstrate that MEX3C deficiency results in lean phenotype and protected mice from diet-induced obesity. Modulating MEX3C activity could be a novel strategy to control obesity and type II diabetes.

### REFERENCES

Agoulnik, A. I., Lu, B., Zhu, Q., Truong, C., Ty, M. T., Arango, N., Chada, K. K. and Bishop, C. E. (2002). A novel gene, Pog, is necessary for primordial germ cell proliferation in the mouse and underlies the germ cell deficient mutation, gcd. Hum Mol Genet 11, 3047-3053.
Beddington, R. S., Morgernstern, J., Land, H. and Hogan, A. (1989). An in situ transgenic enzyme marker for the midgestation mouse embryo and the visualization of inner cell mass clones during early organogenesis. Development 106, 37-46.
Buchet-Poyau, K., Courchet, J., Hir, H. L., Seraphin, B., Scoazec, J.-Y., Duret, L., Domon-Dell, C., Freund, J.-N. and Billaud, M. (2007). Identification and characterization of human Mex-3 proteins, a novel family of evolutionarily conserved RNA-binding proteins differentially localized to processing bodies. Nucl. Acids Res. 35, 1289-1300.
Ciosk, R., DePalma, M. and Priess, J. R. (2006). Translational Regulators Maintain Totipotency in the Caenorhabditis elegans Germline. Science 311, 851-853.
Draper, B. W., Mello, C. C., Bowerman, B., Hardin, J. and Priess, J. R. (1996). MEX-3 Is a KH Domain Protein That Regulates Blastomere Identity in Early C. elegans Embryos. Cell 87, 205-216.
Folch, J., Lees, M. and Stanley, G. H. S. (1957). A SIMPLE METHOD FOR THE ISOLATION AND PURIFICATION OF TOTAL LIPIDES FROM ANIMAL TISSUES. Journal of Biological Chemistry 226, 497-509.
Gesta, S., Tseng, Y.-H. and Kahn, C. R. (2007). Developmental Origin of Fat: Tracking Obesity to Its Source. Cell 131, 242-256.
Guzman, B., Cormand, B., Ribases, M., Gonzalez-Nunez, D., Botey, A. and Poch, E. (2006). Implication of chromosome 18 in hypertension by sibling pair and association analyses: putative involvement of the RKHD2 gene. Hypertension 48, 883-91.
Hauner, H., Schmid, P. and Pfeiffer, E. F. (1987). Glucocorticoids and insulin promote the differentiation of human adipocyte precursor cells into fat cells. J Clin Endocrinol Metab 64, 832-5.
Hirsch, J. and Batchelor, B. (1976). Adipose tissue cellularity in human obesity. Clinics in Endocrinology and Metabolism 5, 299-311.
Hunter, C. P. and Kenyon, C. (1996). Spatial and Temporal Controls Target pal-1 Blastomere-Specification Activity to a Single Blastomere Lineage in C. elegans Embryos. Cell 87, 217-226.
Lu, B., Geurts, A. M., Poirier, C., Petit, D. C., Harrison, W., Overbeek, P. A. and Bishop, C. E. (2007). Generation of rat mutants using a coat color-tagged Sleeping Beauty transposon system. Mamm Genome 18, 338-46.
Moitra, J., Mason, M. M., Olive, M., Krylov, D., Gavrilova, O., Marcus-Samuels, B., Feigenbaum, L., Lee, E., Aoyama, T., Eckhaus, M. et al. (1998). Life without white fat: a transgenic mouse. Genes Dev. 12, 3168-3181.
Musco, G., Stier, G., Joseph, C., Castiglione Morelli, M. A., Nilges, M., Gibson, T. J. and Pastore, A. (1996). Three-dimensional structure and stability of the KH domain: molecular insights into the fragile X syndrome. Cell 85, 237-45.
Pittenger, M. F., Mackay, A. M., Beck, S. C., Jaiswal, R. K., Douglas, R., Mosca, J. D., Moorman, M. A., Simonetti, D. W., Craig, S. and Marshak, D. R. (1999). Multilineage Potential of Adult Human Mesenchymal Stem Cells. Science 284, 143-147.
Reue, K. a. b. c. and Phan, J. a. (2006). Metabolic consequences of lipodystrophy in mouse models. Current Opinion in Clinical Nutrition & Metabolic Care 9, 436-441.
Spalding, K. L., Arner, E., Westermark, P. O., Bernard, S., Buchholz, B. A., Bergmann, O., Blomqvist, L., Hoffstedt, J., Naslund, E., Britton, T. et al. (2008). Dynamics of fat cell turnover in humans. Nature 453, 783-787.
Tang, W., Zeve, D., Suh, J. M., Bosnakovski, D., Kyba, M., Hammer, R. E., Tallquist, M. D. and Graff, J. M. (2008). White fat progenitor cells reside in the adipose vasculature. Science 322, 583-6.
Tontonoz, P., Hu, E. and Spiegelman, B. M. (1994). Stimulation of adipogenesis in fibroblasts by PPAR gamma 2, a lipid-activated transcription factor. Cell 79, 1147-56.
Wang, Y.-X., Lee, C.-H., Tiep, S., Yu, R. T., Ham, J., Kang, H. and Evans, R. M. (2003). Peroxisome-Proliferator-Activated Receptor [delta] Activates Fat Metabolism to Prevent Obesity. Cell 113, 159-170.
Zuk, P. A., Zhu, M., Mizuno, H., Huang, J., Futrell, J. W., Katz, A. J., Benhaim, P., Lorenz, H. P. and Hedrick, M. H. (2001). Multilineage cells from human adipose tissue: implications for cell-based therapies. Tissue Eng 7, 211-28.

1. A method of screening a candidate compound for activity in inhibiting fat deposition in a subject in need thereof and/or treating a condition in a subject in need thereof, comprising:
(a) contacting a candidate compound to a cell that expresses MEX3C protein; and then
(b) detecting a quantity of expression of said MEX3C protein in said cell; a depression in the expression of MEX3C protein in said cell when said candidate compound is contacted thereto as compared to that expressed when said candidate compound is not contacted thereto indicating said compound is active in inhibiting fat deposition and/or treating a condition in a subject in need thereof.

2. The method of claim 1, wherein said cell is in vitro.

3. The method of claim 2, wherein said contacting step is carried out by adding said compound to a media carrying said cell.

4. The method of claim 1, wherein said cell is in vivo in a host animal.

5. The method of claim 4, wherein said contacting step is carried out by orally or parenterally administering said compound to said animal.

6. The method of claim 1, wherein said host cell is a mammalian host cell.

7. The method of claim 5, wherein said host cell is a human, primate, cat, dog, rat, or mouse host cell.

8. The method of claim 1, wherein said cell is a nerve cell, smooth muscle cell, granulosa cell, early spermatogenic cells, or late haploid spermatids.

9. The method of claim 1, wherein said detecting step further comprises at least partially purifying a sample of MEX3C protein from said cell.

10. The method of claim 1, wherein said detecting step further comprises immunologically, spectrophotometrically, or chromatographically detecting said MEX3 C protein.

11. The method of claim 1, wherein said screening comprises screening said compound for activity in treating a disorder selected from the group consisting of obesity, diabetes, syndrome X, glaucoma, hyperlipidemia, hyperglycemia and hyperinsulinemia, vascular conditions associated with elevated sterol and/or stanol levels, arteriosclerotic disease, renal disease, hepatic disease, and metabolic syndrome.

12. The method of claim 1, wherein said compound is selected from the group consisting of oligomers and non-oligomers.

13. A method of screening a candidate compound for activity in inhibiting fat deposition in a subject in need thereof and/or treating a condition in a subject in need thereof, comprising:
(a) contacting a candidate compound to a MEX3C protein in vitro; and then
(b) detecting binding of said MEX3C protein to said candidate compound, binding of said candidate compound to said MEX3C protein indicating said candidate compound is active in inhibiting fat deposition and/or treating a condition in a subject in need thereof.

14. The method of claims 13, wherein said MEX3C protein is mammalian.

15. The method of claim 14, wherein said MEX3C protein is human, primate, cat, dog, rat, or mouse.

16. The method of claim 13, wherein said detecting step is carried out by competitive binding assay.

17. The method of claim 13, wherein said screening comprises screening said compound for activity in treating a disorder selected from the group consisting of obesity, diabetes, syndrome X, glaucoma, hyperlipidemia, hyperglycemia and hyperinsulinemia, vascular conditions associated with elevated sterol and/or stanol levels, arteriosclerotic disease, renal disease, hepatic disease, and metabolic syndrome.

18. The method of claim 13, wherein said compound is selected from the group consisting of oligomers and non-oligomers.

19. A method of screening a subject for a predisposition to a metabolic disorder, comprising:
detecting elevated expression of MEX3C protein in a biological sample from said subject; and
determining said subject is predisposed to a metabolic disorder from said detected elevated expression.

20. The method of claim 19, wherein said metabolic disorder is selected from the group consisting of: obesity, diabetes, syndrome X, glaucoma, hyperlipidemia, hyperglycemia and hyperinsulinemia, vascular conditions associated with elevated sterol and/or stanol levels, arteriosclerotic disease, renal disease, hepatic disease, and metabolic syndrome.

21. The method of claim 19, wherein said biological sample is a blood or tissue sample.

22. The method of claim 19, further comprising at least partially purifying said biological sample.

23. The method of claim 19, wherein said detecting step further comprises immunologically, spectrophotometrically, or chromatographically detecting said MEX3C protein.

24. A method of inhibiting fat deposition in a subject in need thereof, comprising administering said subject an active agent that inhibits the activity and/or expression of the MEX3C protein in said subject in a treatment effective amount.

25. The method of claim 24, wherein said subject is afflicted with obesity, diabetes, syndrome X, glaucoma, hyperlipidemia, hyperglycemia and hyperinsulinemia, vascular conditions associated with elevated sterol and/or stanol levels, arteriosclerotic disease, renal disease, hepatic disease, and metabolic syndrome.

26. The method of claim 24, wherein the active agent is a small molecule, nucleic acid, protein, antibody or antigen-binding fragment thereof.

27. The method of claim 24, wherein the active agent is an RNAi agent.

28. The method of claim 27, wherein the RNAi agent is a siRNA, shRNA, miRNA, dsRNA or ribozyme.


SEQUENCE LISTING (68 sequences)

SEQ ID NO: 1 - DNA, 21 bp, Artificial
  MEX3C target sequence
aagaggacgaggaggaggggg

SEQ ID NO: 2 - RNA, 21 nt, Artificial
  siRNA sequence
gaggacgaggaggaggggguu

SEQ ID NO: 3 - RNA, 21 nt, Artificial
  siRNA sequence
cccccuccuccucguccucuu

SEQ ID NO: 4 - DNA, 21 bp, Artificial
  MEX3C target sequence
aagcggagctggacggagacc

SEQ ID NO: 5 - RNA, 21 nt, Artificial
  siRNA sequence
gcggagcuggacggagaccuu

SEQ ID NO: 6 - RNA, 21 nt, Artificial
  siRNA sequence
ggucuccguccagcuccgcuu

SEQ ID NO: 7 - DNA, 21 bp, Artificial
  MEX3C target sequence
aagcagaggaggaggaccggt

SEQ ID NO: 8 - RNA, 21 nt, Artificial
  siRNA sequence
gcagaggaggaggaccgguuu

SEQ ID NO: 9 - RNA, 21 nt, Artificial
  siRNA sequence
accgguccuccuccucugcuu

SEQ ID NO: 10 - DNA, 21 bp, Artificial
  MEX3C target sequence
aacggggagcaggcggccctg

SEQ ID NO: 11 - RNA, 21 nt, Artificial
  siRNA sequence
cggggagcaggcggcccuguu

SEQ ID NO: 12 - RNA, 21 nt, Artificial
  siRNA sequence
cagggccgccugcuccccguu

SEQ ID NO: 13 - DNA, 21 bp, Artificial
  MEX3C target sequence
aaagagcgtcaacaccaccga

SEQ ID NO: 14 - RNA, 21 nt, Artificial
  siRNA sequence
agagcgucaacaccaccgauu

SEQ ID NO: 15 - RNA, 21 nt, Artificial
  siRNA sequence
ucggugguguugacgcucuuu

SEQ ID NO: 16 - DNA, 21 bp, Artificial
  MEX3C target sequence
aacaccaccgagtgcgtcccg

SEQ ID NO: 17 - RNA, 21 nt, Artificial
  siRNA sequence
caccaccgagugcgucccguu

SEQ ID NO: 18 - RNA, 21 nt, Artificial
  siRNA sequence
cgggacgcacucggugguguu

SEQ ID NO: 19 - DNA, 21 bp, Artificial
  MEX3C target sequence
aaaattaaagcactgagagcc

SEQ ID NO: 20 - RNA, 21 nt, Artificial
  siRNA sequence
aauuaaagcacugagagccuu

SEQ ID NO: 21 - RNA, 21 nt, Artificial
  siRNA sequence
ggcucucagugcuuuaauuuu

SEQ ID NO: 22 - DNA, 21 bp, Artificial
  MEX3C target sequence
aattaaagcactgagagccaa

SEQ ID NO: 23 - RNA, 21 nt, Artificial
  siRNA sequence
uuaaagcacugagagccaauu

SEQ ID NO: 24 - RNA, 21 nt, Artificial
  siRNA sequence
uuggcucucagugcuuuaauu

SEQ ID NO: 25 - DNA, 21 bp, Artificial
  MEX3C target sequence
aaagcactgagagccaagaca

SEQ ID NO: 26 - RNA, 21 nt, Artificial
  siRNA sequence
agcacugagagccaagacauu

SEQ ID NO: 27 - RNA, 21 nt, Artificial
  siRNA sequence
ugucuuggcucucagugcuuu

SEQ ID NO: 28 - DNA, 21 bp, Artificial
  MEX3C target sequence
aagacaaacacgtatatcaag

SEQ ID NO: 29 - RNA, 21 nt, Artificial
  siRNA sequence
gacaaacacguauaucaaguu

SEQ ID NO: 30 - RNA, 21 nt, Artificial
  siRNA sequence
cuugauauacguguuugucuu

SEQ ID NO: 31 - DNA, 21 bp, Artificial
  MEX3C target sequence
aaacacgtatatcaagactcc

SEQ ID NO: 32 - RNA, 21 nt, Artificial
  siRNA sequence
acacguauaucaagacuccuu

SEQ ID NO: 33 - RNA, 21 nt, Artificial
  siRNA sequence
ggagucuugauauacguguuu

SEQ ID NO: 34 - DNA, 21 bp, Artificial
  MEX3C target sequence
aagactcctgttcgtggtgaa

SEQ ID NO: 35 - RNA, 21 nt, Artificial
  siRNA sequence
gacuccuguucguggugaauu

SEQ ID NO: 36 - RNA, 21 nt, Artificial
  siRNA sequence
uucaccacgaacaggagucuu

SEQ ID NO: 37 - DNA, 21 bp, Artificial
  MEX3C target sequence
aagagcccatttttgttgtca

SEQ ID NO: 38 - RNA, 21 nt, Artificial
  siRNA sequence
gagcccauuuuuguugucauu

SEQ ID NO: 39 - RNA, 21 nt, Artificial
  siRNA sequence
ugacaacaaaaaugggcucuu

SEQ ID NO: 40 - DNA, 21 bp, Artificial
  MEX3C target sequence
aaggaaagaagatgttgccat

SEQ ID NO: 41 - RNA, 21 nt, Artificial
  siRNA sequence
ggaaagaagauguugccauuu

SEQ ID NO: 42 - RNA, 21 nt, Artificial
  siRNA sequence
auggcaacaucuucuuuccuu

SEQ ID NO: 43 - DNA, 21 bp, Artificial
  MEX3C target sequence
aaagaagatgttgccatggcc

SEQ ID NO: 44 - RNA, 21 nt, Artificial
  siRNA sequence
agaagauguugccauggccuu

SEQ ID NO: 45 - RNA, 21 nt, Artificial
  siRNA sequence
ggccauggcaacaucuucuuu

SEQ ID NO: 46 - DNA, 21 bp, Artificial
  MEX3C target sequence
aagatgttgccatggccaaaa

SEQ ID NO: 47 - RNA, 21 nt, Artificial
  siRNA sequence
gauguugccauggccaaaauu

SEQ ID NO: 48 - RNA, 21 nt, Artificial
  siRNA sequence
uuuuggccauggcaacaucuu

SEQ ID NO: 49 - DNA, 21 bp, Artificial
  MEX3C target sequence
aaaagagagatcctctcagct

SEQ ID NO: 50 - RNA, 21 nt, Artificial
  siRNA sequence
aagagagauccucucagcuuu

SEQ ID NO: 51 - RNA, 21 nt, Artificial
  siRNA sequence
agcugagaggaucucucuuuu

SEQ ID NO: 52 - DNA, 21 bp, Artificial
  MEX3C target sequence
aagagagatcctctcagctgc

SEQ ID NO: 53 - RNA, 21 nt, Artificial
  siRNA sequence
gagagauccucucagcugcuu

SEQ ID NO: 54 - RNA, 21 nt, Artificial
  siRNA sequence
gcagcugagaggaucucucuu

SEQ ID NO: 55 - DNA, 21 bp, Artificial
  MEX3C target sequence
aaacaaaaatgggcctgccct

SEQ ID NO: 56 - RNA, 21 nt, Artificial
  siRNA sequence
acaaaaaugggccugcccuuu

SEQ ID NO: 57 - RNA, 21 nt, Artificial
  siRNA sequence
agggcaggcccauuuuuguuu

SEQ ID NO: 58 - DNA, 21 bp, Artificial
  MEX3C target sequence
aaaaatgggcctgccctggga

SEQ ID NO: 59 - RNA, 21 nt, Artificial
  siRNA sequence
aaaugggccugcccugggauu

SEQ ID NO: 60 - RNA, 21 nt, Artificial
  siRNA sequence
ucccagggcaggcccauuuuu

SEQ ID NO: 61 - DNA, 20 bp, Artificial
  RT-PCR primer
gaaggaagagcgtcaacacc

SEQ ID NO: 62 - DNA, 20 bp, Artificial
  RT-PCR primer
gccatggcaacatcttcttt

SEQ ID NO: 63 - DNA, 23 bp, Artificial
  Lac-Z cDNA probe
gccagggttttcccagtcacgac

SEQ ID NO: 64 - protein, 20 aa, Artificial
  Rabbit anti-MEX3C antibody-generating peptide sequence
DPSGNMKTQRRGSQPSTPRC

SEQ ID NO: 65 - RNA, 25 nt, Artificial
  siRNA sequence
ccuagcaguugacucuccugccuuu

SEQ ID NO: 66 - DNA, 37 bp, Artificial
  Gene trap splice junction sequence
attgtcggacgccagggtcccaggtcccgaaaaccaa

SEQ ID NO: 67 - DNA, 31 bp, Artificial
  Gene trap splice junction sequence
ttgtcggacgccaggttgcctgactccccgt

SEQ ID NO: 68 - DNA, 29 bp, Artificial
  Gene trap splice junction sequence
gccgggaagctagagttgtaaaattaaag